(19) United States
(12) Patent Application Publication
(10) Pub. No.: US 2018/0229045 A1
(43) Pub. Date: Aug. 16, 2018

(54) Charging Coil Holding Device for an Implantable Medical Device with Movable Charging Coil

(71) Applicant: Boston Scientific Neuromodulation Corporation, Valencia, CA (US)

(72) Inventors: Matthew L. McDonald, Pasadena, CA (US); Daniel Aghassian, Glendale, CA (US)

(21) Appl. No.: 15/894,593

(22) Filed: Feb. 12, 2018

Related U.S. Application Data
(60) Provisional application No. 62/458,295, filed on Feb. 13, 2017.

(51) Int. Cl. A61N 1/378; A61N 1/372

(52) U.S. Cl. CPC A61N 1/3787; A61N 1/37217

(57) ABSTRACT

A holding device is disclosed which can hold a charging coil for providing power to an Implanted Medical Device (IMD). To assist with charging coil to IMD alignment, movable elements are provided to allow a patient to move the charging coil on or in the holding device while the holding device stays stationary against the patient. The holding device can accommodate an integrated external charger or a charging coil assembly that is controlled by a separate charging controller, and the charging controller may also be affixed in or on the holding device. Movable elements preferably allow the charging coil to be moved parallel to the long axis of the holding device (x), although they may also allow the charging coil to be moved perpendicularly to the long axis (y), or to be rotated (θ) around an axis pointing away from the holding device.

### CROSS REFERENCE TO RELATED APPLICATIONS

This is a non-provisional application of U.S. Provisional Patent Application Ser. No. 62/458,295, filed Feb. 13, 2017, to which priority is claimed, and which is incorporated by reference in its entirety.

### FIELD OF THE TECHNOLOGY

The present disclosure relates to an external charging system for an implantable medical device.

### INTRODUCTION

Implantable stimulation devices deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators (DBS) to treat motor and psychological disorders, and other neural stimulators to treat urinary incontinence, sleep apnea, shoulder subluxation, etc. The description that follows will generally focus on the use of the technology within a spinal cord stimulation (SCS) system, such as that disclosed in U.S. Pat. No. 6,516,227. However, the present disclosure may find applicability with any implantable medical device (IMD) or in any IMD system.

As shown in FIG. 1, a SCS system includes an Implantable Pulse Generator (IPG) 10 (hereinafter, and more generically, IMD 10), which includes a biocompatible device case 12 formed of titanium for example. The case 12 typically holds the circuitry and battery 14 necessary for the IMD 10 to function. The IMD 10 is coupled to electrodes 16 via one or more electrode leads 18 (two of which are shown). The proximal ends of the leads 18 are coupled to the IMD 10 at one or more lead connectors 20 fixed in a header 22, which can comprise an epoxy for example. There are sixteen electrodes (E1-E16) in the illustrated example, although the number of leads and electrodes is application specific and therefore can vary. In an SCS application, two electrode leads 18 are typically implanted on the right and left side of the dura within the patient's spinal column. The proximal ends of the leads 18 are then tunneled through the patient's tissue 35 (FIG. 5) to a distant location, such as the buttocks, where the IMD case 12 is implanted, at which point they are coupled to the lead connectors 20.

An IMD 10 is typically supported by and communicates with one or more external devices, and FIGS. 2 and 3 provide examples of such devices. FIG. 2 depicts an external controller 40 for the IMD 10. The external controller 40 is used to establish a bi-directional wireless data link with the IMD 10, and is typically used to send or adjust the therapy settings the IMD 10 will provide to the patient. If the IMD 10 is an IPG 10 as depicted in FIG. 1, such therapy settings may include which electrodes 16 are active to issue therapeutic current pulses; whether such electrodes sink or source current (i.e., electrode polarity); the duration, frequency, and amplitude of the pulses, etc., which settings together comprise a stimulation program for the IMD 10. External controller 40 can also act as a receiver of data from the IMD 10, such as various data reporting on the IMD's status and the level of the IMD's battery 14.

As shown in FIG. 2, external controller 40 is typically configured in a hand-held, portable housing 42, and powered by an internal battery (not shown). The external controller 40 includes a Graphical User Interface (GUI) similar to that used for a cell phone, including buttons 44 and a screen 46, and may have other interface aspects as well, such as a speaker. While an external controller 40 is typically a device custom built by the manufacturer of the IMD 10 and dedicated in its functionality to IMD communications, external controller 40 may also comprise a general purpose, freely programmable mobile device having suitable wireless communication functionality, such as a smart cell phone. In this case, a Medical Device Application (MDA) can be executed on the mobile device to configure the device's GUI for use as an IMD external controller, and to allow for control and monitoring of the IMD 10. See, e.g., U.S. Patent Application Publication 2015/0073498.

Both the external controller 40 and IMD 10 have antennas to effectuate the bi-directional data communication link, and such antennas can come in different forms and operate pursuant to different protocols or communication standards. In one example, both these devices can includes coil antennas, with IMD 10 including an antenna coil 28a inside its case 12, and external controller 40 including an antenna coil 50a inside its housing 42. Such antennas, which may comprise a plurality of turns of wire, may communicate by near-field magnetic induction, as is well known. The IMD 10 and external controller 40 may also include radio-frequency (RF) antennas 28b and 50b, which communicate by far-field electromagnetic waves, and which may comprise wires, slots, or patches for example. Although shown inside the case 12, the IMD 10's data antenna 28a or 28b can also be located in its header 22.

FIG. 3 depicts an external charger 60 for an IMD 10, see U.S. Pat. No. 8,682,444, which is used to recharge the IMD 10's rechargeable battery 14 or to provide continuous power to the IMD 10 should it lack a battery. External charger 60 produces an AC magnetic charging field 65 (FIG. 5) from a charging coil 68a, which again may comprise a plurality of turns. The user interface of the external charger 60 may be simple compared to the external controller 40. For example, the external charger 60 may lack a screen. Instead, the external charger 60 may simply include an on/off button 64 for magnetic charging field 65 generation, and a light emitting diode (LED) 66 to indicate when the magnetic field is being generated. Although not shown, external charger 60 may also include a speaker useful to indicate alignment between the external charger and the IMD 10, as is well known. See, e.g., U.S. Patent Application Publication 2013/0096651. External charger 60 is also typically hand-held and portable, with its electrical components, including the charging coil 68a, integrated within a housing 62. External charger 60 may additionally include data communication abilities, and may effectively comprise a charger and controller combined into a single housing, as disclosed in U.S. Pat. No. 8,335,569 for example.

FIG. 4 shows another external charger 70 that may be used to charge IMD 10, which is disclosed for example in U.S. Pat. Nos. 8,498,716 and 8,463,392. This device includes a charging coil assembly 75, which includes a charging coil 68b within a housing 76. The charging coil assembly 75 couples to a charging controller 80 via a cable 74, for example, using a connector 72 on the cable and a port 82 on the charging controller. Cable 74 can also be hardwired to the charging controller 80. Although not shown, the charging controller 80 includes a user interface to allow a patient to charge his IMD 10—i.e., to produce a magnetic field 65 (FIG. 5)—and may also allow feedback relevant to IMD charging to be indicated (such as charger-to-IMD alignment). The charging controller 80 may comprise a device specifically dedicated to charging functionality, as disclosed in U.S. Patent Application Publication 2007/0060980. Alternatively, the charging controller 80 may comprise the external controller 40 described earlier (FIG. 2), in which case the charging controller 80 would include charging functionality in addition to data-communication functionality. As shown, a hole 78 may be present in the housing 76 of the assembly 75 in the center of the charging coil 68b.

Regardless whether an integrated external charger 60 (FIG. 3) or a charging coil assembly 75 (FIG. 4) is used, FIG. 5 shows in cross section generation of the magnetic charging field 65 from charging coils 68a or 68b of those devices. The magnetic field 65 is preferably in the range of 300 kHz or less, and passes through the patient's tissue 35 to induce a current in the IMD 10's charging coil 24. Although well-known processing circuitry in the IMD 10 is not shown, this AC current is rectified to a DC level and then used to charge the IMD battery 14 or to provide continuous power to the IMD if it lacks a battery.

Because charging the battery 14 in the IMD 10 may take some time, or because charging must be supplied continuously, it is desired to hold charging coil 68a or 68b in close proximity to and in alignment with the IMD 10 during a charging session when the magnetic charging field 65 is produced. As disclosed in U.S. Patent Application Publication 2014/0025140 and shown here in FIG. 6, this can occur using an external charger holding device 90, such as a belt 92, which accommodates the integrated external charger 60 of FIG. 3. In an SCS application in which the IMD 10 is typically implanted in the upper portion of a patient's buttocks, the belt 92 fastens around a patient's waist, and can be secured by a fastening device 94, such as a buckle, clasp, snaps, Velcro, etc. The belt 92 includes a pouch 96, which is generally located on the belt 92 in a rearward position where the IMD 10 is implanted in the patient. An opening 98 in the belt 92 allows the external charger 60 to be inserted into the pouch 96, such that the external changer 60 is, like the pouch 96, generally aligned with the IMD 10. Once placed in the pouch 96, the patient can press the on/off switch 64 (FIG. 3) on the external charger 60 to begin a charging session—i.e., to produce magnetic charging field 65—or the user can turn the charger on before inserting it in the pouch 96. Affixing the external charger 60 to the patient using holding device 90 allows the patient to move or walk while using the external charger 60, and thus can charge his implant “on the go.” See also U.S. Patent Application Publication 2012/0012630, describing another belt for an external charger.

Another external charger holding device 100 disclosed in U.S. Patent Application Publication 2016/0301239 and useable with external charger 70 of FIG. 4 is shown in FIG. 7. This holding device 100 is similar to the holding device 90 in several respects which aren't repeated. Differences stems from the fact that external charger 70 has a separate, non-integrated charger controller 80/charging coil assembly 75 structure. The charging coil assembly 75 appears at a first location 95 on the belt 92, and may be permanently contained within the material of the belt 92, although the assembly 75 an also be removable from the belt. First location 95 is preferably rearward to be generally aligned to the IMD 10's location. Cable 74 between the charging coil assembly 75 and the charger controller 80 (which again may comprise a dedicated external controller or a mobile device for example) proceeds at least partially through the material of the belt 92. The charger controller 80 is placed at a foreward second location 97 on the belt 92, and may be removable from and insertable into the belt 92 through an opening 99. When inserted into the belt 92, the charger controller 80 is preferably connected to the cable 74 using the connector 72/port 82 described earlier.

Regardless whether holding device 90 or 100 is used in conjunction with external charger 60 or 70, the inventors are concerned that these holding devices can make it difficult to align the charging coil 68a or 68b with the IMD 10. In this regard, and returning to FIG. 5, charging of the IMD 10 will be most efficient—i.e., take the least amount of time, or require the least amount of energy—when the charging coil 68a or 68b is centered with respect to the IMD 10, or more particularly when the central axis 101 around which coil 68a or 68b is wound is collinear with the central axis 103 of the IMD 10's charging coil 24. If these axes 101 and 103 are offset (x), coupling between the coils will be poor: charging will either be slow; or the energy of the magnetic field 65 must be increased at the external charger 60 or 70 to charge at the same rate. This is a particular problem for the holding devices 90 and 100 of FIGS. 6 and 7. While these holding devices both allow a patient to charge on the go, patient movement can cause misalignment between the charging coil 68a/68b in the charger and charging coil 24 in the IMD 10, in particular because the belt 92 of the holding device will tend to move around the patient. In other words, and as shown in FIGS. 6 and 7, such movement causes an offset (x) similar to that depicted in FIG. 5.

Movement of the belt 92 around the patient, and resulting misalignment between the charging coil 68a or 68b and the IMD 10, is inconvenient for the patient. First, as alluded to above and as explained in many of the references cited herein, external chargers such as 60 and 70 will typically monitor charging coil-to-IMD alignment, and when they become misaligned, will indicate this status to the patient. Such indication may come in the form of a beeping sound intended to alert the patient that he must try to move his external charger (or charging coil assembly) into better alignment with the IMD 10; if realignment is successful this indication will cease. Trying to establish (or reestablish) alignment of holding devices 90 or 100 can be difficult. First, the location of the IMD 10 in the patient's tissue may be difficult to determine, as may the locations of the charging coil 68a or 68b (e.g., the pouch 96 (FIG. 6) or first location 95 (FIG. 7)), particularly when these are behind the patient and outside of view. Second, it may not be easy for the patient to simply rotate the holding device 90 or 100 around his waist, as it is usually tightly fastened. The fastening device 94 may therefore need to be unfastened, which create further uncertainty as to the location of the charging coil because the belt 92 will tend to slump downward, thus adding further uncertainty about relative charger-to-IMD positioning and thus how alignment can be achieved.

### BRIEF DESCRIPTION OF THE DRAWINGS

FIG. 1 shows an implantable pulse generator (IPG) type of implantable medical device (IMD), in accordance with the prior art.

FIGS. 2 and 3 show external devices for an IMD, including an external controller (FIG. 2) and an integrated external charger (FIG. 3), in accordance with the prior art.

FIG. 4 shows an external charger for an IMD having a charging controller and a charging coil assembly connected by a cable, in accordance with the prior art.

FIG. 5 shows in cross section the charging coils in an external charger and the IMD, and shows generation of a magnetic field to provide power to the IMD, in accordance with the prior art.

FIG. 6 shows a holding device used to hold the external charger of FIG. 3 in general alignment with an IMD implanted in a patient, in accordance with the prior art.

FIG. 7 shows another holding device used to hold the external charger of FIG. 4 in general alignment with an IMD implanted in a patient, in accordance with the prior art.

FIGS. 8A and 8B show a first example of a holding device for an external charger, in which an integrated external charger is moveable within the device parallel to a long axis of the holding device, and including one or more movable elements (e.g., one or more cables) to affect such movement.

FIGS. 9A and 9B show a second example of a holding device for an external charger, in which an integrated external charger is moveable within the device by a cable connected to the external charger by Velcro.

FIGS. 10A and 10B show a third example of a holding device for an external charger, in which an integrated external charger is moveable within the device by one or more cables and a pulley that allows the outward ends of cable(s) to exit on a same side of the holding device.

FIGS. 11A-11C show a fourth example of a holding device for an external charger, in which an integrated external charger is moveable within the device by a loop cable, a pulley, and a rotatable wheel.

FIGS. 12A-12E show a fifth example of a holding device for an external charger, in which an integrated external charger is moveable on the outside of the device.

FIGS. 13A and 13B show a sixth example of a holding device for an external charger, in which an integrated external charger is moveable on a rail on the outside of the device.

FIGS. 14A and 14B show a seventh example of a holding device for an external charger, in which one or more movable elements allow a charging coil assembly to be moved within the device parallel to a long axis of the holding device, and in which the holding device also optionally holds a charging controller.

FIGS. 15A and 15B show an eighth example of a holding device for an external charger, in which a charging coil assembly is moveable within the device, and in which the charging controller can be clipped to the holding device.

FIGS. 16A and 16B show a ninth example of a holding device for an external charger, in which a charging coil assembly is moveable on the outside of the device.

FIGS. 17A and 17B show a tenth example of a holding device for an external charger, in which a charging coil assembly is moveable on a rail on the outside of the device.

FIG. 18 shows an eleventh example of a holding device for an external charger, in which movable elements allow the charging coil to be moved in a direction perpendicular to the long axis of the holding device.

FIGS. 19A and 19B show a twelfth example of a holding device for an external charger, in which movable elements allow the charging coil to be moved angularly around an axis pointing away from the holding device.

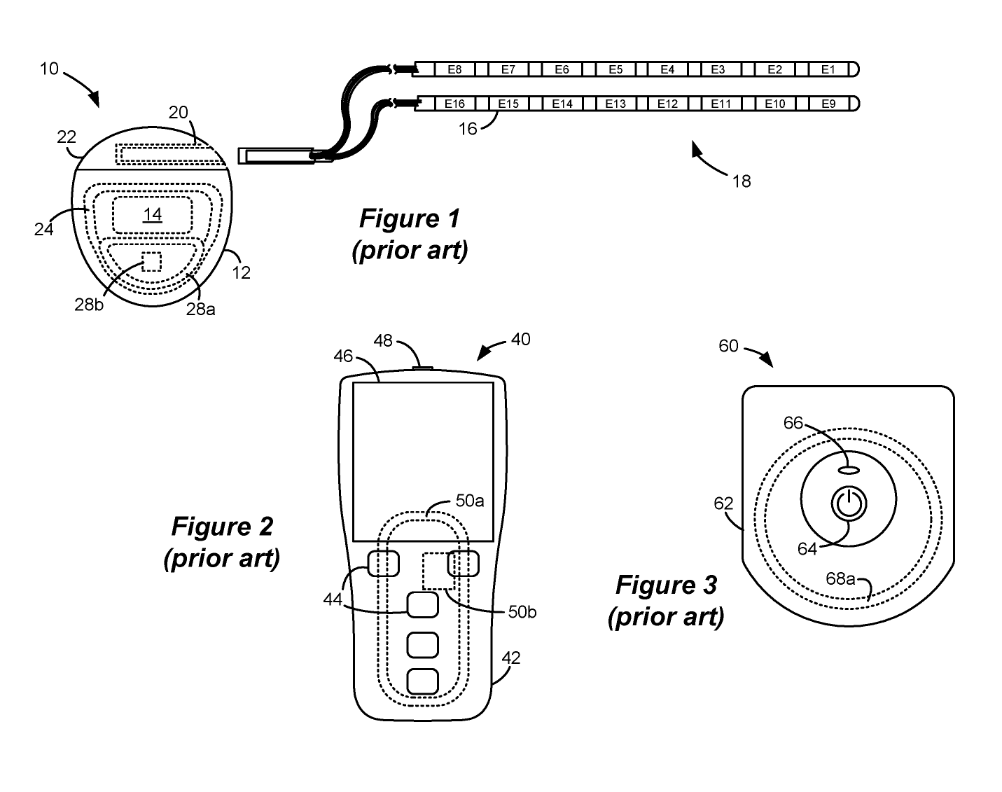

### DETAILED DESCRIPTION

To address the concerns discussed in the Background, a holding device such as a belt is disclosed which can hold an external charger for providing power to an Implanted Medical Device (IMD). To assist with charging coil to IMD alignment, movable elements are provided to allow a patient to move a portion of the external charger that has the charging coil (e.g., the entire integrated charger or a charging coil assembly) on or in the holding device while the holding device stays stationary against the patient. Movable elements can include cables that a patient can pull, although other elements are possible. The holding device can accommodate an integrated external charger or a charging coil assembly that is controlled by a separate charging controller, and the charging controller may also be affixed in or on the holding device. Movable elements preferably allow the charging coil to be moved parallel to the long axis of the holding device (x), although they may also allow the charging coil to be moved perpendicularly to the long axis (y), or to be rotated (0) around an axis pointing away from the holding device.

Subsequent figures show different examples of holding devices that can operate as just described, with each having different features. Some examples also use features included in earlier examples, and these are generally referred to using the same element numerals introduced earlier. While various combinations of features are shown in the various holding device examples, other combinations of features are possible, and it should be understood that the various features disclosed can be used in a holding device in any combination, even though all useful combinations are not shown.

The subsequent examples of holding devices, like those described in the Background (90, 100), can comprise a belt such that intended to be worn around a patient's waist, which is particular useful in a Spinal Cord Stimulation (SCS) application in which an IMD 10 is implanted behind the patient in his/her buttocks. However, this is not strictly necessary and instead where the holding device might be worn on a patient can depend on where an IMD 10 is implanted in a patient. For example, in a Deep Brain Stimulation (DBS) application, a patient's IMD 10 may be implanted in the chest under the collarbone for example, in which case holding device 150 would be worn as a harness or sling, such as around a patient's neck. See, e.g., U.S. Pat. No. 8,626,297.

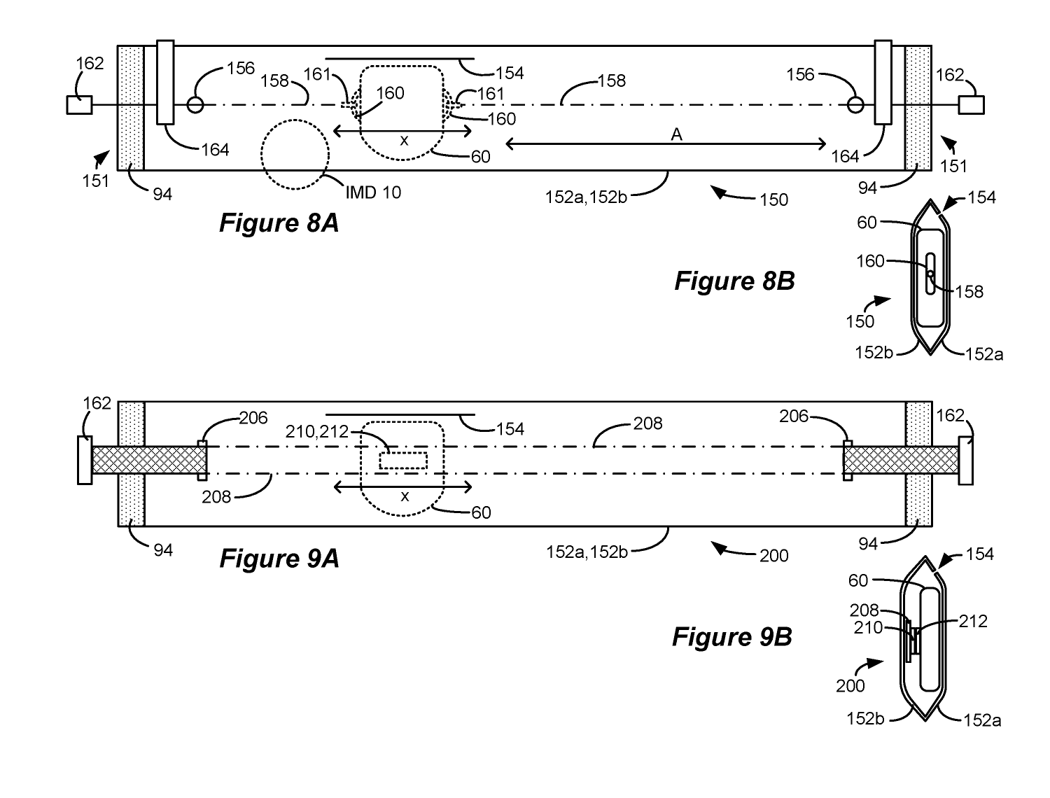

A first example of an improved holding device 150 is shown in a plan view in FIG. 8A and in a cross sectional view in FIG. 8B. The holding device 150 is shown in FIG. 8A fully opened and laid flat, thus showing the device's long axis A. The holding device 150 can be fastened around a patient using a fastening device 94 appearing at one or both ends 151 of the device, which as discussed earlier can comprise a buckle, clasp, snaps, Velcro, etc. Although the holding device 150 has a long axis A, the holding device need not be perfectly straight or perfectly parallel to this long axis, although it is so shown in FIG. 8A for ease of drawing.

The holding device is formed in FIGS. 8A and 8B of two pieces of material: an outer piece 152a that faces away from the patient when worn, and an inner piece 152b that faces towards and touches the patient (or the patient's clothing). Outer and inner pieces 152a and 152b may be formed of various cloth, plastic, or rubber materials, or combinations of these, and may be adhered to one another by sewing, heat sealing, and the like. Outer and inner pieces 152a and 152b may also be formed of a single piece which is folded. In the example shown, the outer and inner pieces 152a and 152b extend the entire length of the holding device 150 along its long axis A, but in practice need only extend so far on the holding device 150 as the external charger 60 is meant to move (x), as explained subsequently.

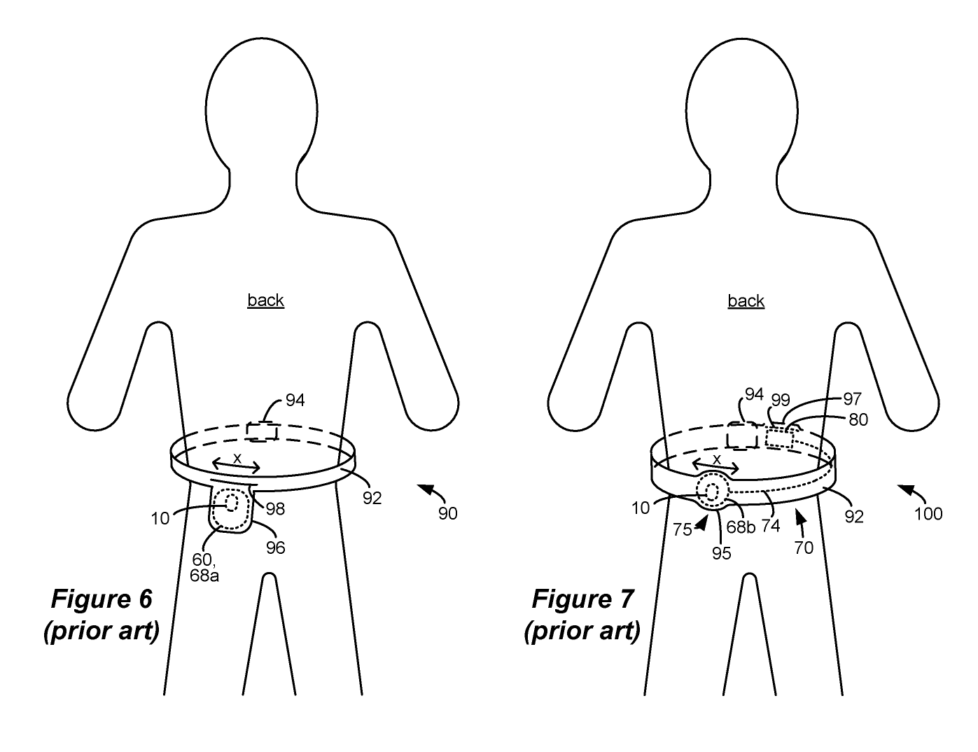

The holding device 150 of FIGS. 8A and 8B is envisioned as useful to hold an integrated external charger 60 containing a charging coil 68a and control electronics such as described earlier (FIG. 3). In this regard, and similar to holding device 90 discussed in the Background (FIG. 6), the holding device 150 includes an opening 154 into which the integrated external charger 60 may be inserted. The opening 154 as shown is formed in the outer piece 152a, but may also be formed for example at the junction of pieces 152a and 152b. Once the external charger 60 is inserted, notice in the cross section of FIG. 8B that the external charger 60 is located between the outer and inners pieces 152a and 152b of the holding device 150.

Further shown in FIG. 8A are movable elements that allow a user to move (x) the external charger 60 within the holding device 150 parallel to its long axis (A). In FIG. 8A, the movable elements comprise one or more cables 158 that are connectable to the external charger 60. Specifically, a patient may connect inwards ends of the cables 158 to the external charger 60 through the opening 154. In the example shown, the inward ends of the cables 158 include hooks 161 that connect with loops 160 on the external charger 60. However, there are many variations as to how cable(s) 158 may be connected to the external charger 60. For example, if two cables 158 are used as shown, their inward ends may include Velcro, which can connect to Velcro on the external charger 60. A single cable 158 may also be used, which passes along and is preferably connected to the back of the external charger 60. (This is further discussed with respect to FIGS. 9A and 9B). Clamps, clips, snaps and other like devices may also be used to connect the cable(s) 158 to the external charger 60. Cable(s) 158 may comprise a rope or thread, but may take other forms as well, as discussed further below. For example, cable(s) 158 may comprise toothed pinions controlled with toothed racks able to promote movement.

The outward ends of the cable(s) 158 proceed between the outer and inner pieces 152a and 152b and preferably exit the outer piece 152b at openings 156 proximate to each end of the holding device 150. If desired, the outward ends of the cable(s) 158 can include pull tabs 162, which may be sized larger than the openings 156 so that the outward ends will not slip back into the space between the outer and inner pieces 152a and 152b.

With the cable(s) 158 so connected to the external charger 60, and when the patient has affixed the holding device 150 to himself using fastening device 94, the outward ends of the cable(s) 158 (e.g., pull tabs 162) will be proximate to each other, and in an SCS application will generally be at the front of the patient. The patient may then pull on the right pull tab 162 to move the external charger 60 to the right (x) through the outer and inners pieces 152a and 152b, and may similarly pull the left pull tab 162 to move the external charger 60 to the left.

This assists the patient in aligning the external charger 60 and in particular its charging coil 68a (FIG. 3) with the IMD 10 that the external charger 60 is powering, and addresses shortcomings associated with holding device 90 (FIG. 6) described in the Background. If the external charger 60 is indicating misalignment—e.g., if it is beeping as described earlier—the patient need not attempt to rotate the holding device 150 around his waist, nor unfasten the fastening device 94: that is, inner piece 152b can continue to be held firmly against the patient, and preferably will not move relative to the patient. Instead, the user may pull the left and right pull tabs 162 to move the external charger 60 through the holding device 150 from left to right until alignment is established and the misalignment indicator ceases. For example, pulling left pull tab 162 in FIG. 8A will move the external charger 60 to the left, eventually bringing the external charger 60 into better alignment with the underlying IMD 10, which should eventually cause the misalignment indicator to cease. Again, although the external device 60 is movable as just described, the holding device 150 otherwise remains stationary relative to the patient.

Optionally, holding device 150 may include one or more clamps 164 designed to hold the external charger 60 in place within the holding device 150 once it has been moved by the patient into better alignment with the IMD 10. The clamps are shown generically 164, and can be pinched by the patient around the outsides of the outer and inner pieces 152a and 152b to pinch the cable(s) 158 between them. Clamps 164 are not shown in subsequent examples of holding devices for clarity, although they could be used with such other examples as well. Other means may also be used to hold the charging coil in place after it has been moved inside a holding device.

FIGS. 9A and 9B illustrate another example of a holding device 200, which like holding device 150 can accommodate an integrated external charger 60. In this example, holding device 200 is again formed of outer and inner pieces 152a and 152b, and again the external charger 60 is inserted into the holding device 200 through opening 154. However, in this example, there is only one cable 208 that connects to the external charger 50, and the cable 208 is flat like a ribbon. The cable 208 includes a piece of Velcro 210 connectable to Velcro 212 connected to the back of the external charger 60. Again, other means of connection are possible as discussed earlier. Because the cable 208 is flat, openings 206 are made larger to allow exit of the cable 158 from the outer piece 152a, and again pull tabs 162 may be sized to prevent the cable 208 from slipping back into the holding device 200 through openings 206. As before, once the external charger 60 is connected to the cable 208, pull tabs 162 allow the external charger 60 to be moved (x) within the holding device 200 through outer and inner pieces 152a and 152b parallel to long axis A, thus allowing a patient to conveniently establish alignment between the external charger 60 and the IMD 10 without moving the holding device 200 relative to the patient.

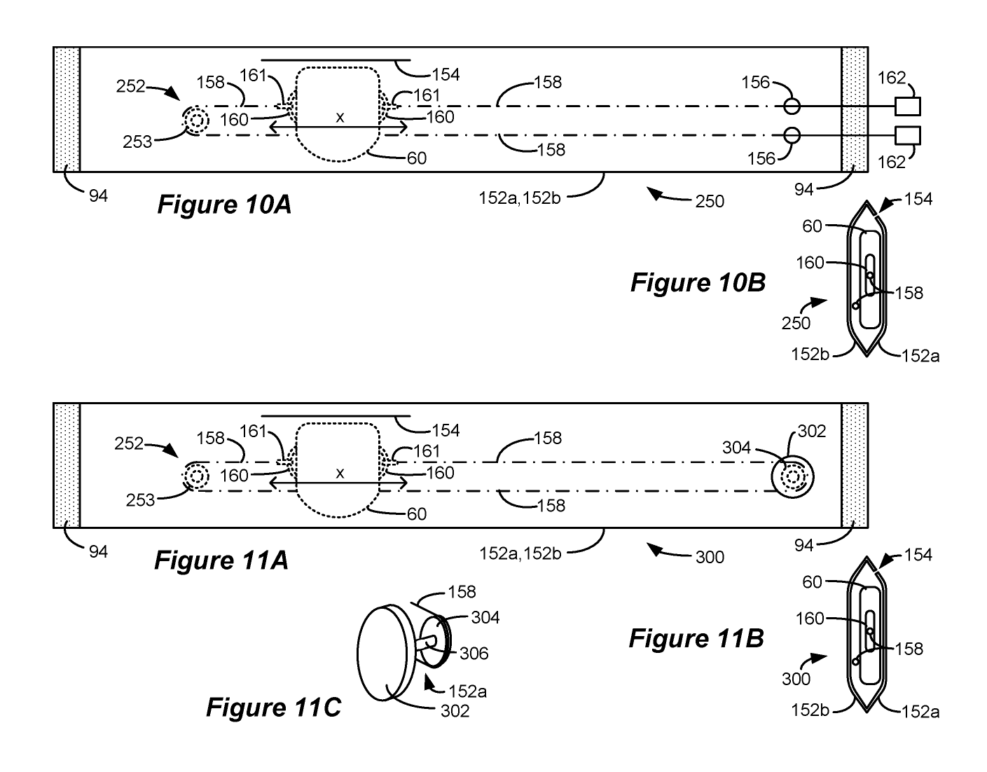

FIGS. 10A and 10B illustrate another example of a holding device 250, which again can accommodate an integrated external charger 60. In this example, holding device 250 is again formed of outer and inner pieces 152a and 152b, and again the external charger 60 is inserted into the holding device 250 through opening 154 and connected to cable(s) 158. (A hook 161 and loop 160 connection is shown, but other connection means previously described could be used in this and other depicted examples, even if not explicitly shown. Further, the use of two cables 158 are shown, but a single cable 158 could be used in this and other subsequent examples, even if not specifically shown).

In this example, outward ends of the cable(s) 158 exit on the same (right) side of the holding device 250. A pulley 252 is included within the outer and inners pieces 152a and 152b on the left side of the external charger 60. The pulley 252 is preferably connected to the inner piece 152a that faces the patient, such as by stitching or heat sealing, includes a rotating grooved wheel 253 in the space between inner and outer pieces 152a and 152b for capturing the cable 158. The cable 158 is looped around the grooved wheel 253 of the pulley, thus allowing the cable to return in the reverse direction so that it exits on the same (right) side of the holding device 250 as already mentioned. As shown in the cross section of FIG. 10B, the returning cable 158 can slip freely behind (as shown) on in front of the external charger 60. Outward ends of cable(s) 158 can emerge from the inside of the holding device 250 through outer piece 152a through openings 156 as explained before. Two openings 156 are shown, although both outward ends of cable(s) 158 could emerge through a single opening 156. Pull tabs 162 are provided at the outward ends of the cable, such that pulling one (top) tab 162 causes the external charger 60 to move in one direction (right) through the holding device 250, while pulling the other (bottom) moves it in the other direction (left).

FIGS. 11A and 11B illustrate another example of a holding device 300, which again can accommodate an integrated external charger 60. In this example, holding device 300 is again formed of outer and inner pieces 152a and 152b, and again the external charger 60 is inserted into the holding device 300 through opening 154 and connected to the cable(s) 158. However, in this example, the cable 158 is formed as a single loop. A pulley 252 with a rotating grooved wheel 253 is provided as in FIG. 10A on one side of the external charger 60. The other side of the external charger 60 includes a rotatable wheel 302 to assist in moving the external charger 60. As shown in FIG. 11C, the rotatable wheel 302 is located outside the outer piece 152a and so can be turned by a patient, and is preferably positioned toward one of the edges of the holding device 300 so that it is in front of the patient (in an SCS application). Inside the outer piece 152a (i.e., between the outer and inner pieces 152a and 152b) resides another rotating grooved wheel 304 rigidly connected to the rotatable wheel 302 by an axle 306 (which axle 306 proceeds through a hole (not shown) in the outer piece 152a). Although not shown, the rotating grooved wheel 304 can be connected to the inner piece 152b similarly to the manner in which the pulley 252 is connected to allow the grooved wheel 403 to freely turn. In this manner, the patient can turn the rotatable wheel 302, which turns the grooved wheel 304, and causes the cable 158 to spin around the grooved wheels 253 and 304. This moves the external charger 60 connected to the cable(s) 158 within the holding device 300, similarly to the manner described earlier with respect to FIGS. 10A and 10B (note the similarity between the cross sectional views of FIGS. 10B and 11B).

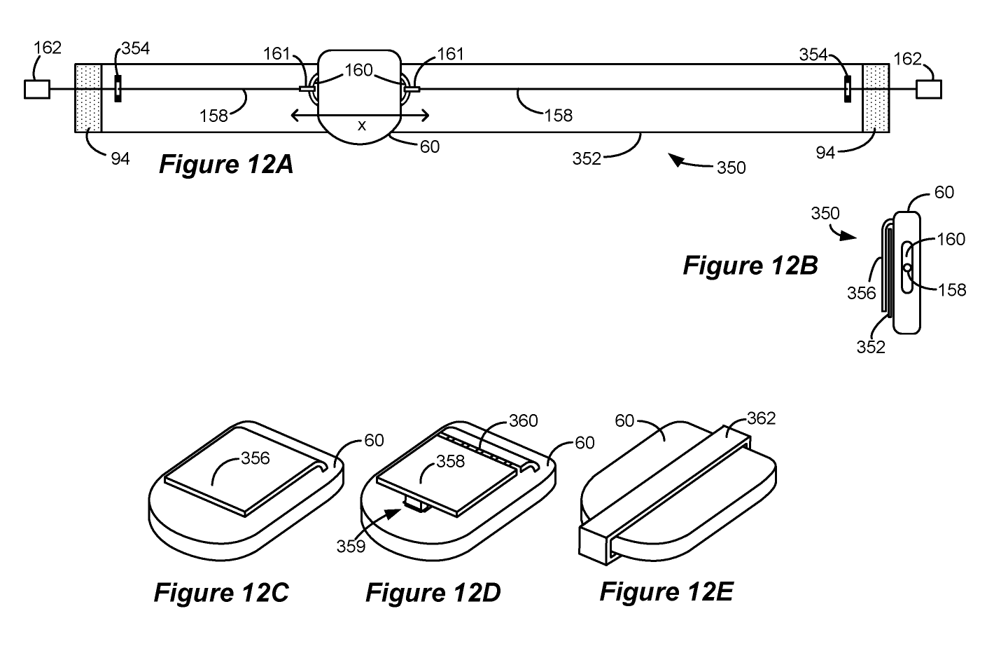

FIGS. 12A and 12B illustrate another example of a holding device 350, which again can accommodate an integrated external charger 60. In this example, holding device 350 comprises only a single material piece 352 over which the external charger 60 rides when moved by a patient. As before, the external charger 60 is connected to one or more cables 158, and one or more restraining members 354 can be coupled to the outer side of the piece 352 to guide cable(s) 158 toward outward ends of the holding device, and to keep pull tabs 162 in position.

The external charger 60 is connected to the piece 352 in a manner that allows it to move along the outer side of the piece 352 that faces away from the patient when worn, and various manners in which this may occur are illustrated in FIGS. 12C-12E. FIG. 12C shows that the external charger 60 includes a clip 356, which is preferably on the external charger's backside. Clip 356 allow the external charger 60 to hang on the piece 352 and to slide left or right over the piece 365 (x) when either the left or right pull tabs 162 is pulled. Use of a clip 356 is shown in the cross section of FIG. 12B, although other means of slidably connecting the external charger 60 to the piece 365 (FIGS. 12D and 12E) could also be used.

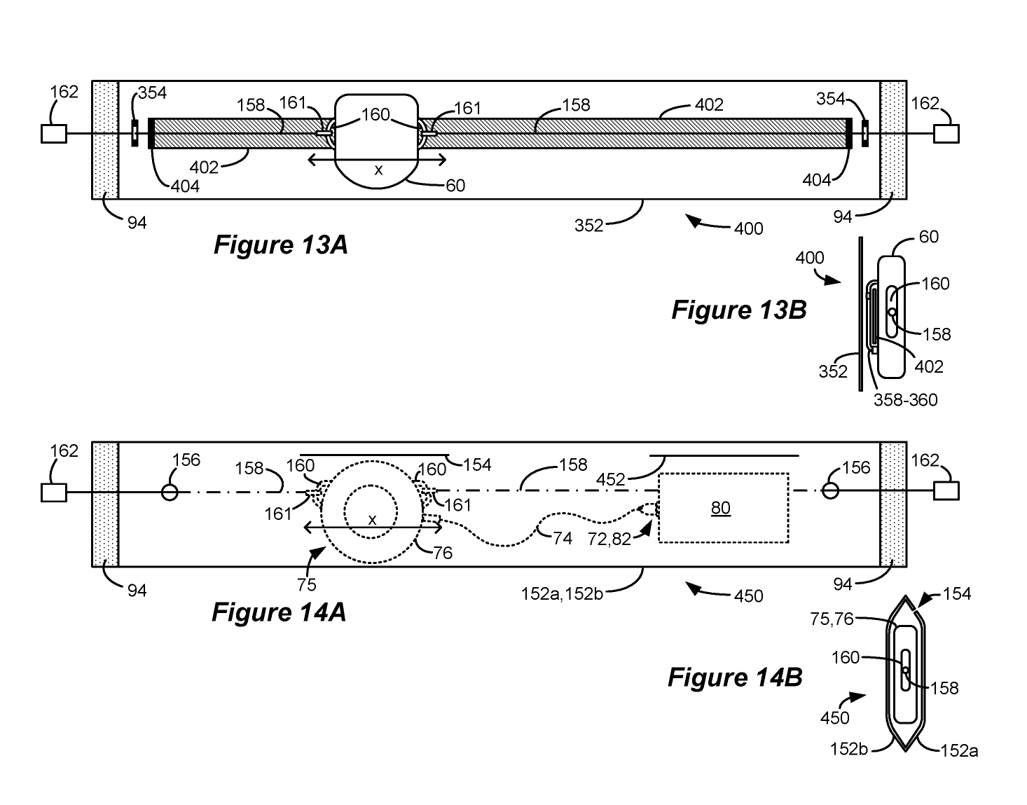

FIG. 12D shows an external charger 60 with a hinged latch, including a hinge 360, a clip 358, and a latch 359 capable of connecting (e.g., snapping) to the external charger 60. With this configuration, the patient can open the latch 359, place the piece 352 in the clip 358, and close the latch 359 to enclose the piece 352 within the clip 358. This example may be preferable to that shown in FIG. 12C because the piece 352 is effectively trapped by the external charger 60, thus preventing the external charger 60 from slipping away from the piece 352 as the external charger moves over the piece 352. FIG. 13B, discussed subsequently, shows use of this means of slidably retaining external device to a holding device in a different context.

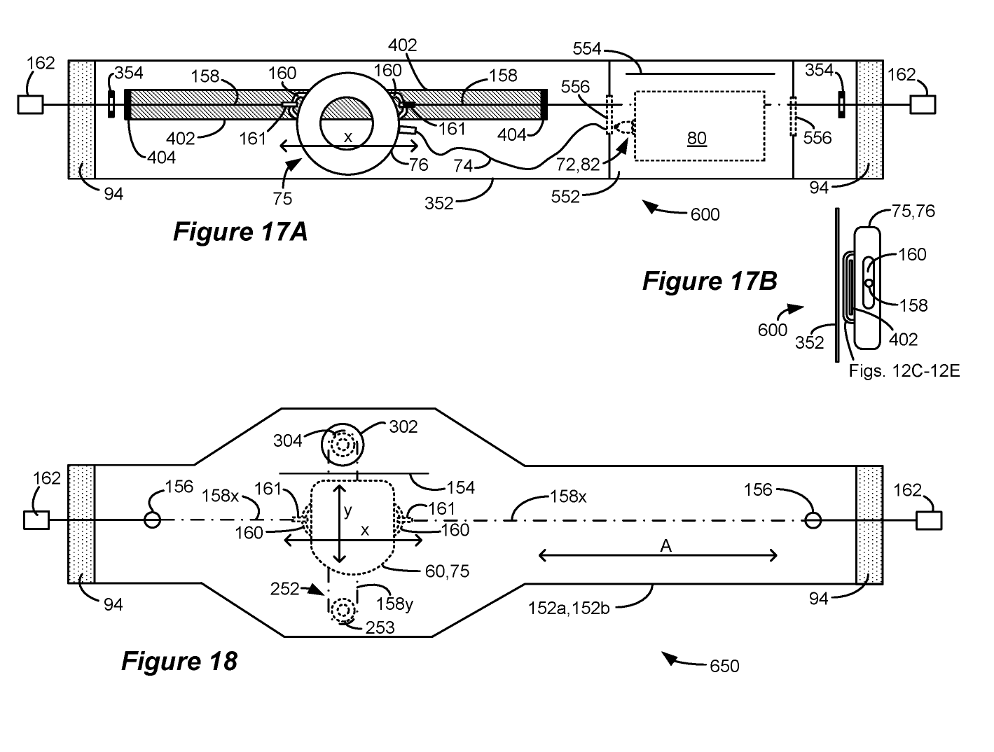

FIG. 12E shows use of a clamp 362 to slidably hold the external charger 60 to the piece 352. Clamp 362 can comprise a structure separate from the external charger 60, which can be positioned over the external charger 60 and piece 352 to trap the piece 352 and allow the external charger 60 to slide with respect to the piece 352. Clamp 362 may for example comprise a generally hard rubber that is deformable by the patient to snap the clamp 362 around the piece 352 and at least a portion of the external charger 60. FIGS. 16A and 16B, discussed subsequently, shows use of clamp 362 in a different context.

FIGS. 13A and 13B illustrate another example of a holding device 400, which again can accommodate an integrated external charger 60. In this example, holding device 400 again comprises only a single piece 352. However, a rail 402 is attached to the outer side of piece 352 (using stiches 404), and in this example the external charger 60 slides over the rail 402 instead of the piece 352 when moved by a patient. As before, the external charger 60 is connected to one or more cables 158, and one or more restraining members 354 are preferably coupled to the outer side of the piece 352 to guide cable(s) 158 toward outward ends of the holding device, and to keep pull tabs 162 in position. The external charger 60 can be slidably connected to the rail 402 using any of the sorts of connection means described earlier in FIGS. 12C-12E to slidably connect the charger to the piece 352. Used of a hinged latch 358-360 (FIG. 12D) is shown in the cross section of FIG. 13B as just one example.

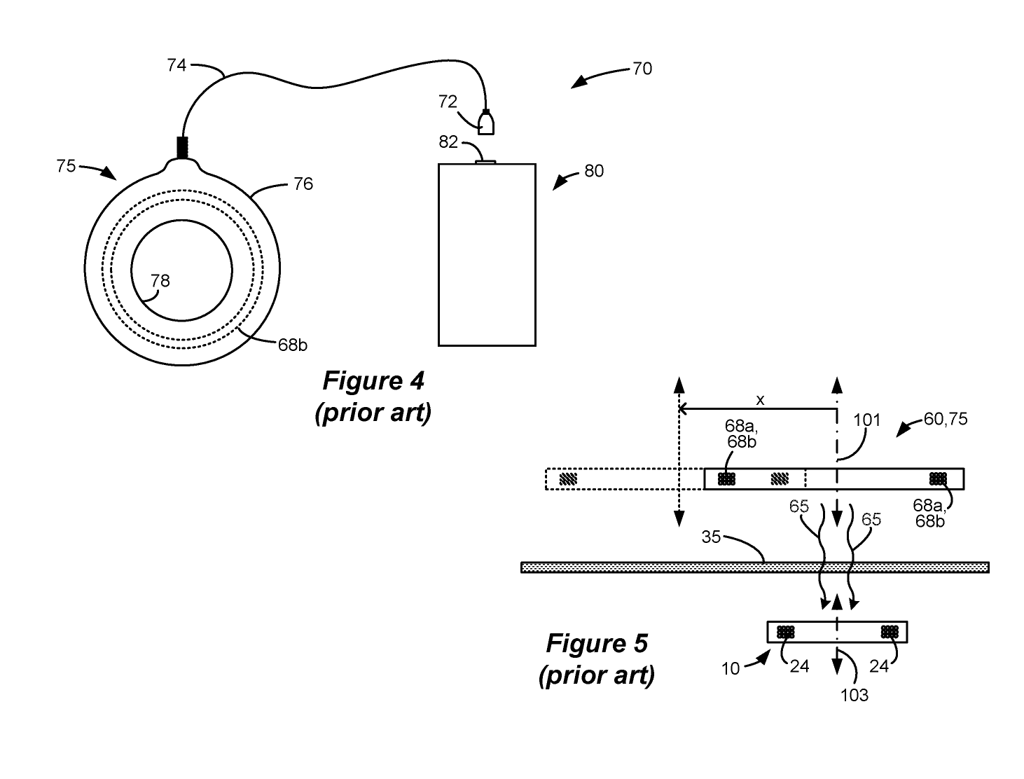

To this point, a holding device useable with an integrated external charger 60 (FIG. 3) has been shown, but other holding devices can be used with an external charger 70 having a separate charging coil assembly 75 and charging controller 80, as described earlier with respect to FIG. 4. Examples of these holding devices are shown in FIGS. 14A-17B, and all generally include movable elements to allow a patient to move the charging coil assembly 75 and its charging coil 68b within or on the holding device to assist in achieving alignment with the patient's IMD 10. Manners in which the charging controller 80 may be conveniently connected to or held by the holding devices are also shown. However, a holding device need not include the capability to retain a charging controller 80; a patient may decide to retain his charging controller 80 in a different manner not involving the holding device, such as in a pants pocket for example. Many of the features of the subsequent examples of holding devices useable with a charging coil assembly 75 are similar to those illustrated earlier, and thus are described more briefly.

FIGS. 14A and 14B show a holding device 450 useable with a charging coil assembly 75 and further include means for holding the charging controller 80. In this example, outer and inner pieces 152a and 152b are again used. The charging coil assembly 75 is placed in opening 154 and its housing 76 is connected to one or more cables 158 as before. (Again, hooks 161 and loops are shown 160 in this and subsequent examples, but this is just for simplicity and other means could be used, including means using a single cable). The holding device 450 can also hold the charging controller 80, which is inserted in an opening 452, such that the charging controller 80 like the charge coil assembly 75 is housed between the outer and inner pieces 152a and 152b. The assembly 75's cable 74 also proceeds between the outer and inner pieces 152a and 152b so it can be connected to the charging controller 80 at connector/port 72/82 through opening 452. However, because only the charging coil assembly 75 is connected to the cable(s) 158, only it is moved (x) within the holding device 450 when the cable(s) are pulled; charging controller 80 stays in place, with cable(s) 158 moving past it (e.g., behind it) when pulled.

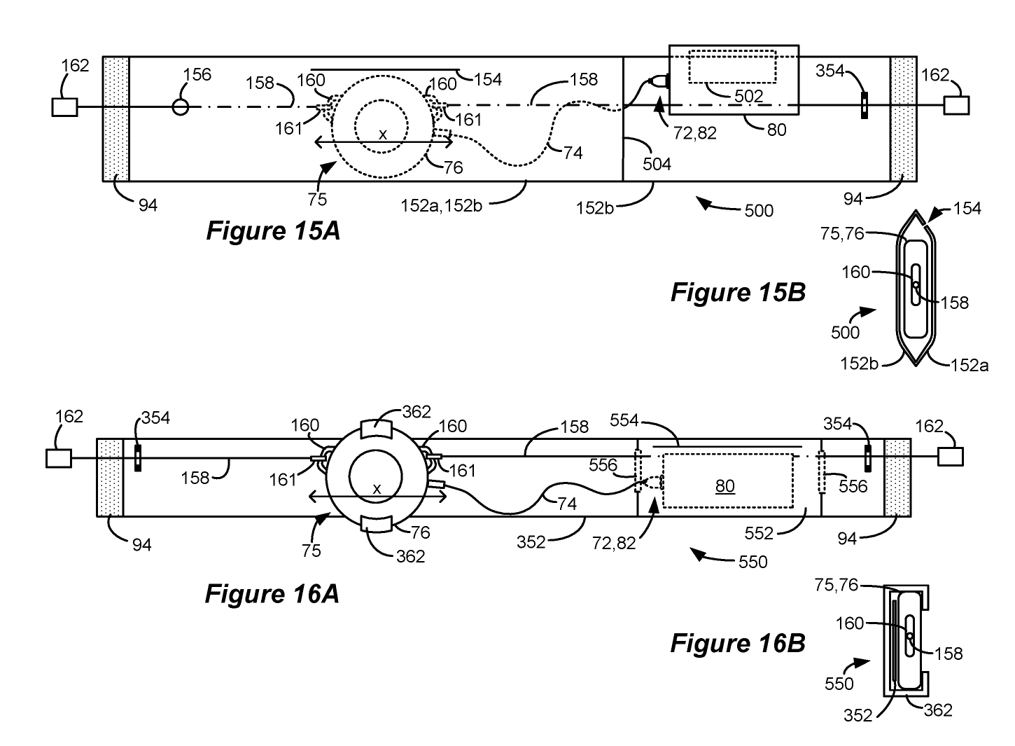

FIGS. 15A and 15B show a holding device 500 useable with a charging coil assembly 75. In this example, only the portion of the holding device 500 that holds the charging coil assembly 75 includes outer and inner pieces 152a and 152b. Outside of this portion, the holding device 500 comprises only inner piece 152b, and this portion can be used as a place to attach the charging controller 80. The charging controller 80 can attach to the inner piece 152a in many manners, but in this example, the external controller 80 includes a clip 502. An opening 504 may be included at the end of the outer piece 152a to allow the cable 74 of the charging assembly 75 to exit and be connected to the charging controller 80. Otherwise, movement (x) of the charging coil assembly 75 within the holding device 500 occurs similarly to that discussed with respect to FIGS. 14A and 14B, and using the movable elements.

FIGS. 16A and 16B show a holding device 550 that is analogous to that shown earlier in FIGS. 12A-12E, although in this example, it is the charging coil assembly 75 that is slidably affixed to the single piece 352 and movable by a patient using cable(s) 158. In this example, a clamp 362 (FIG. 12E) is used, and the manner in which the piece 352 slips between the clamp 362 and the charging coil assembly 75 is shown in cross section in FIG. 16B, although again other means could be used (FIGS. 12C, 12D). In this example a pouch 552 is included to hold the charging controller 80, which may be inserted into the pouch 552 though an opening 554. The pouch 552 may be formed of another (outer) material piece in addition to (inner) piece 352. Sides of the pouch 552 include openings 556 to allow the cable 158 to slidably pass across the otherwise stationary charging controller 80, and to allow passage of the assembly 75's cable 74 into the pouch 552, where it can be connected to the charge controller 80 (72/82).

FIGS. 17A and 17B show a holding device 660 that is analogous to that shown earlier in FIGS. 13A-13E, although in this example, it is the charging coil assembly 75 that is slidably affixed to the rail 402 that is attached to the single piece 352. Other features of FIGS. 17A and 17B has been previously explained.

To this point, examples of holding devices have been shown that allow a charging coil 68a or 68b in an integrated external charger 60 or charging coil assembly 75 to be moved in or on a holding device in a direction (x) parallel to the long axis A of the device. However, in other examples, the charging coil can be moved in other directions, which may also be important to establishing alignment with a patient's IMD 10.

For example, the holding device 650 shown in FIG. 18 allows a charging coil to be moved in a direction y perpendicular to the holding device's long axis A. The holding device 650 as illustrated has basic similarity to the holding device 150 of FIGS. 8A and 8B, and allows for movement in parallel direction x as guided by one or more cable(s) 158x as described earlier. However, this is merely one example, and any of the other holding devices previously discussed could be used to provide parallel movement. In addition, holding device 650 includes movable elements to provide movement of the external charger 60 or charging coil assembly 75 in a perpendicular y direction while the holding device 650 otherwise remains stationary relative to the patient as previously described. Any of the previously introduced movable elements could be used, but in the depicted example a loop cable 158y is used similar to that used in holding device 300 of FIGS. 11A-11C, and includes a pulley 252 with its grooved wheel 253, and rotatable wheel 302 with its grooved wheel 304. Although not shown, cable 158y would be connected to the external charger 60 or charging coil assembly 75, such as by Velcro as already described. (If a charging coil assembly 75 is used in conjunction with a charging controller 80, the charging controller 80 could affix to the holding device 650 in any of the manners previously mentioned, although this is not shown in FIG. 18). The cable 158y, pulley 252, and rotatable wheel 302 are positioned perpendicularly to the long axis A such that when a patient turns the rotatable wheel 302, the cable 158y will turn, thus moving the external charger 60 up or down (y). The ability to move the external charge 60 in a y direction is a useful to assist in fine tuning alignment with the IMD 10. Although not shown, holding device 650 may not include x-direction movable elements, but instead may include only y-direction movable elements.

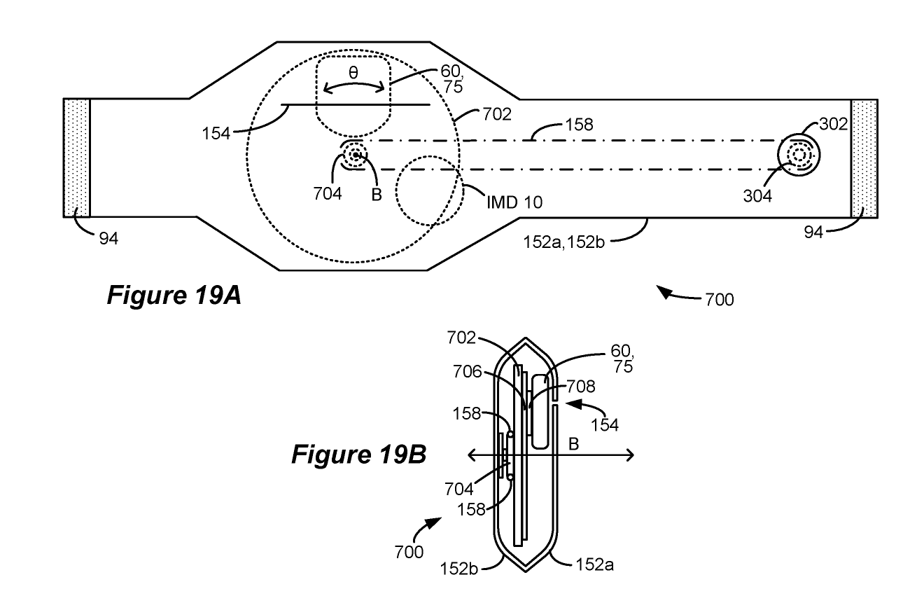

FIGS. 19A and 19B show another example of a holding device 700 which allows a charging coil to be moved angularly (θ) around an axis B pointing away from the holding device while the holding device otherwise remains stationary relative to the patient as previously described. In this example, the external charger 60 or charging coil assembly 75 is connected to a rotating plate 702 within the holding device 700. As best seen in FIG. 19B, this connection can be established through opening 154 using Velcro 706 attached to the outward facing portion of the rotating plate 702, and Velcro 708 connected to the back of the charging coil 68a or 68b in either external charger 60 or charging coil assembly 75. Connection to the rotating plate 702 however may be made by other means. The rotating plate 702 is rigidly connected to a grooved wheel 704, which is in turn rotatably connected by an axle connected to the inner piece 152b. A loop cable 158 is provided in the groove in the grooved wheel 704 and grooved wheel 304. As described earlier, grooved wheel 304 is connected to a rotatable wheel 302 which a patient can turn, and which is preferably proximate to the front of the patient to facilitate patient access.

When the rotatable wheel 302 is turned, the grooved wheel 304 will turn, thus moving the cable 158 and turning the grooved wheel 704. This turns the rotating plate 702, which turns the external charger 60 or charging coil assembly 75 (θ) within the holding device 700—e.g., between the outer and inner pieces 152a and 152b. As earlier examples illustrate however, such movement of the device 60/75 may occur on, not within the holding device. Because the external charger 60 or charging coil assembly 75 is preferably connected to the rotating plate 702 off center of axis B, such movement (θ) will rotate the device 60/75 around axis B. Thus, the patient may turn the rotatable wheel 302 to try and establish better alignment between the device 60/75 and the patient's IMD 10. For example, and as shown in FIG. 19A, if the patient turns the rotatable wheel 302 clockwise, the device 60/75 will eventually be rotated about 120 degrees clockwise, thus establishing better alignment between the device 60/75 and the IMD 10.

Although not shown, cable 158 may not be necessary in an example of a holding device 700 such as shown in FIGS. 19A and 19B. For example, the rotatable wheel 302 may be rigidly connected to the rotating plate 702 at axis B. Thus, the patient could merely turn the rotatable wheel 302 to turn the plate 702 and the device 60/75 (although the patient may need to reach behind himself to do so, e.g., in a SCS application).

Although particular embodiments have been shown and described, it should be understood that the above discussion is not intended to limit the present disclosure to these embodiments. It will be obvious to those skilled in the art that various changes and modifications may be made without departing from the spirit and scope of the present disclosure. Thus, the present disclosure is intended to cover alternatives, modifications, and equivalents that may fall within the spirit and scope of the claims.

What is claimed is:

1. A holding device for an external charger configured to provide wireless power to a patient's implantable medical device (IMD), the holding device comprising:
at least one piece configured to be worn by a patient, wherein the at least one piece has a long axis, wherein first and second ends of the at least one piece are configured to be connected to each other to affix the at least one piece stationary against the patient; and
at least one movable element configured to be connected to a portion of the external charger that includes a charging coil configured to provide the wireless power,
wherein the at least one movable element is controllable by the patient to move the portion of the external charger relative to the at least one piece of the holding device while the at least one piece stays stationary against the patient,
wherein the at least one movable element is accessible by the patient at a front of the patient when the at least one piece is affixed against the patient.

2. The holding device of claim 1, wherein the at least one movable element is configured to move the portion parallel to the long axis.

3. The holding device of claim 1, wherein the at least one movable element is configured to move the portion perpendicular to the long axis.

4. The holding device of claim 1, wherein the at least one movable element is configured to move the portion angularly around an axis pointing away from the at least one piece.

5. The holding device of claim 1, wherein the at least one piece comprises an outer piece that faces away from the patient when worn and an inner piece that faces towards the patient when worn, and wherein the at least one movable element is configured to move the portion between the outer and inner pieces.

6. The holding device of claim 5, further comprising an opening configured to allow the portion to be positioned between the outer and inner pieces, and configured to allow the patient to connect the portion to the at least one movable element.

7. The holding device of claim 1, wherein the at least one piece comprises a single piece, and wherein the at least one movable element is configured to move the portion on an outer side of the piece that faces away from the patient when worn.

8. The holding device of claim 7, wherein the portion includes a clip that hangs on the piece.

9. The holding device of claim 8, wherein the portion includes a latch configured to trap the piece within the latch.

10. The holding device of claim 7, further comprising a clamp separate from the external charger, wherein the clamp is positionable over the portion and the piece to trap the piece within the clamp.

11. The holding device of claim 1, further comprising a rail attached to an outer side of the at least one piece that faces away from the patient when worn, wherein the rail is configured for attachment to the portion, and wherein the at least one movable element is configured to move the portion along the rail.

12. The holding device of claim 1, wherein the at least one movable element comprises at least one cable connected to the portion.

13. The holding device of claim 12, wherein the at least one cable comprises a single ribbon.

14. The holding device of claim 12, wherein the at least one cable has outward ends proximate to the first and second ends of the at least one piece, wherein the outward ends are pullable by the patient to move the portion.

15. The holding device of claim 12, wherein the at least one cable comprises outward ends proximate to only the first end of the at least one piece, wherein the outward ends are pullable by the patient to move the portion.

16. The holding device of claim 12, wherein the at least one cable comprises a single loop cable, and further comprising a pulley and a rotatable wheel, wherein the loop cable is connected to the pulley and rotatable wheel such that the rotatable wheel is turnable by the patient to move the portion.

17. The holding device of claim 1, wherein the portion comprises a housing of an integrated external charger.

18. The holding device of claim 1, wherein the portion comprises a housing of a charging coil assembly, and wherein the external charger further comprises a charging controller connected to the charging coil assembly by a cable.

19. The holding device of claim 18, wherein the holding device is configured to hold the charging controller.

20. A method for holding an external charger using a holding device, wherein the external charger is configured to provide wireless power to a patient's implantable medical device (IMD), the method comprising in any order:
affixing at least one piece of the holding device stationary against the patient;
connecting at least one movable element of the holding device to a portion of the external charger that includes a charging coil configured to provide the wireless power; and
controlling the at least one movable element at a front of the patient to move the portion of the external charger relative to the at least one piece of the holding device while the at least one piece stays stationary against the patient.
